(19) United States
(12) Patent Application Publication
(10) Pub. No.: US 2020/0013617 A1
(43) Pub. Date: Jan. 9, 2020

(54) SUBSTRATE TREATMENT METHOD, SUBSTRATE TREATMENT SYSTEM AND DIRECTED SELF-ASSEMBLING MATERIAL

(71) Applicant: JSR CORPORATION, Tokyo (JP)

(72) Inventors: Hiroyuki KOMATSU, Tokyo (JP); Miki TAMADA, Tokyo (JP); Hitoshi OSAKI, Tokyo (JP); Motohiro SHIRATANI, Tokyo (JP); Tomoki NAGAI, Tokyo (JP)

(73) Assignee: JSR CORPORATION, Tokyo (JP)

(21) Appl. No.: 16/504,581

(22) Filed: Jul. 8, 2019

(30) Foreign Application Priority Data
Jul. 9, 2018 (JP) 2018-129735

(51) Int. Cl. H01L 21/02; C23C 16/40; C23C 16/455; C07C 255/09; C07C 255/34; C07C 255/31

(52) U.S. Cl. CPC H01L 21/02304; H01L 21/02172; H01L 21/0228; C23C 16/40; C07C 2601/14; C07C 255/09; C07C 255/34; C07C 255/31; C07C 2601/16; C23C 16/45525

(57) ABSTRACT

A substrate treatment method includes: overlaying a film on a surface of a substrate which includes a first region including a metal atom in a surface layer thereof, using a directed self-assembling material which contains a compound having no less than 6 carbon atoms and including at least one cyano group. After the overlaying, the film on a region other than the first region is removed. After the removing, a pattern principally containing a metal oxide is formed by an Atomic Layer Deposition process or a Chemical Vapor Deposition process on the region other than the first region, of the surface of the substrate.

### CROSS-REFERENCE TO RELATED APPLICATIONS

The present application claims priority to Japanese Patent Application No. 2018-129735, filed Jul. 9, 2018, the contents of which are incorporated herein by reference in their entirety.

### BACKGROUND OF THE INVENTION

### Field of the Invention

The present invention relates to a substrate treatment method, a substrate treatment system and a directed self-assembling material.

### Discussion of the Background

Further miniaturization of semiconductor devices has been accompanied by a demand for a technique of forming a fine pattern having a line width of less than 30 nm. However, it is technically difficult to form such a fine pattern by conventional methods employing lithography, due to optical factors and the like.

To this end, a bottom-up technique, as generally referred to, has been contemplated for forming a fine pattern. As the bottom-up technique, in addition to a method employing directed self-assembly of a polymer, a method for selectively modifying a substrate having a surface layer that includes fine regions has been recently studied. The method for selectivity modifying the substrate requires a material enabling easy and highly selective modification of surface regions, and various materials have been investigated for such use (see Japanese Unexamined Patent Application, Publication No. 2016-25315; Japanese Unexamined Patent Application, Publication No. 2003-76036; ACS Nano, 9, 9, 8710, 2015; ACS Nano, 9, 9, 8651, 2015; Science, 318, 426, 2007; and Langmuir, 21, 8234, 2005).

### SUMMARY OF THE INVENTION

According to an aspect of the present invention, a substrate treatment method includes: overlaying a film on a surface of a substrate which includes a first region including a metal atom in a surface layer thereof, using a directed self-assembling material which contains a compound having no less than 6 carbon atoms and including at least one cyano group. After the overlaying, the film on a region other than the first region is removed. After the removing, a pattern principally containing a metal oxide is formed by an Atomic Layer Deposition process or a Chemical Vapor Deposition process on the region other than the first region, of the surface of the substrate.

According to another aspect of the present invention, a substrate treatment system includes a mechanism for overlaying a film, a mechanism for removing the film, and a mechanism for forming a pattern. The mechanism for overlaying a film overlays the film on a surface of a substrate which includes a first region including a metal atom in a surface layer thereof, using a directed self-assembling material which includes a compound having no less than 6 carbon atoms and including at least one cyano group. The mechanism for removing the film removes the film on a region other than the first region, after the overlaying. The mechanism for forming a pattern forms the pattern principally including a metal oxide by an Atomic Layer Deposition process or a Chemical Vapor Deposition process on the region other than the first region, of the surface of the substrate, after the removing.

According to further aspect of the present invention, a directed self-assembling material includes a compound having no less than 6 carbon atoms and including at least one cyano group.

### BRIEF DESCRIPTION OF THE DRAWINGS

FIG. 1 is a schematic view illustrating selectively forming a directed self-assembling film on a surface of a metal substrate from a directed self-assembling material of an embodiment; and

FIG. 2 is a schematic view illustrating a blocking performance for metal oxide formation by an ALD process.

### DESCRIPTION OF EMBODIMENTS

According to an embodiment of the invention made for solving the aforementioned problems, a substrate treatment method includes: overlaying a film on a surface of a substrate which comprises a first region comprising a metal atom in a surface layer thereof, using a directed self-assembling material which comprises a compound having no less than 6 carbon atoms and comprising at least one cyano group; after the overlaying, removing the film on a region other than the first region; and after the removing, forming a pattern principally comprising a metal oxide by an ALD (Atomic Layer Deposition) process or a CVD (Chemical Vapor Deposition) process on the region other than the first region, of the surface of the substrate.

According to other embodiment of the present invention made for solving the aforementioned problems, a substrate treatment system includes: a mechanism for overlaying a film on a surface of a substrate which comprises a first region comprising a metal atom in a surface layer thereof, using a directed self-assembling material which comprises a compound having no less than 6 carbon atoms and comprising at least one cyano group; a mechanism for removing the film on a region other than the first region, after the overlaying; and a mechanism for forming a pattern principally comprising a metal oxide by an ALD process or a CVD process on the region other than the first region, of the surface of the substrate, after the removing.

According to still other embodiment of the present invention made for solving the aforementioned problems, a directed self-assembling material contains a compound having no less than 6 carbon atoms and including at least one cyano group.

The substrate treatment method and the substrate treatment system of the embodiments of the present invention enable a treatment for selectively modifying a substrate surface to be executed through achieving superior blocking performance for metal oxide formation in a hydrophobilized region by an ALD process or a CVD process. The directed self-assembling material of the embodiment of the present invention is capable of conveniently and highly selectively hydrophobilizing the surface of the substrate having a region which includes a metal atom in the surface layer thereof, whereby the hydrophobilization treatment enables a superior blocking performance for metal oxide formation by an ALD process or a CVD process to be achieved. Therefore, the substrate treatment method, substrate treatment system and directed self-assembling material can be suitably used for working processes of semiconductor devices, and the like, in which microfabrication is expected to be further in progress hereafter.

Hereinafter, embodiments of the substrate treatment method, the substrate treatment system and the directed self-assembling material will be described in detail.

### Directed Self-Assembling Material

The directed self-assembling material contains a compound having no less than 6 carbon atoms and including at least one cyano group (hereinafter, may be also referred to as “compound (A)” or “(A) compound”). The directed self-assembling material may contain (B) a solvent as a favorable component, in addition to the compound (A), and within a range not leading to impairment of the effects of the present invention, may also contain other optional component(s). Each component will be described below.

(A) Compound

The compound (A) is a compound having no less than 6 carbon atoms and including at least one cyano group.

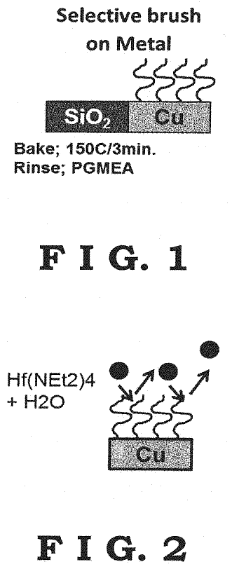

Due to containing the compound (A), the directed self-assembling material is capable of conveniently and highly selectively hydrophobilizing the surface of the substrate having a region which includes a metal atom in the surface layer thereof, whereby the hydrophobilization treatment enables a superior blocking performance for metal oxide formation by an ALD process or a CVD process to be achieved. Although not necessarily clarified and without wishing to be bound by any theory, the reason for achieving the effects described above due to the directed self-assembling material having the aforementioned constitution is inferred as in the following, for example. Specifically, it is considered that the compound (A) can selectively interact with a metal surface by way of its cyano group, and the compound (A) can be oriented by interaction with each other through van der Waals force due to the number of carbon atoms being no less than 6, thereby consequently enabling the metal surface to be highly selectively hydrophobilized. The film formed from such a directed self-assembling material (hereinafter, may be also referred to as “directed self-assembling film”) is believed to achieve superior blocking performance for metal oxide formation by an ALD process or a CVD process on particular, resulting from an aggregation structure, etc., thereof. According to the directed self-assembling material, the directed self-assembling film is formed selectively on the surface of the substrate having a region (first region) including a metal atom in a surface layer thereof as shown in FIG. 1, and the region where the directed self-assembling film is formed achieves the blocking performance for the metal oxide formation by an ALD process as shown in FIG. 2.

The number of the cyano group(s) in the compound (A) is preferably 1 to 10, more preferably 1 to 6, still more preferably 1 to 4, particularly preferably 1 to 3, more particularly preferably 1 or 2, and most preferably 2.

The lower limit of the number of carbon atoms of the compound (A) may be 6, preferably 7, more preferably 8, still more preferably 9, and particularly preferably 10. The upper limit of the number of carbon atoms is preferably 50, more preferably 40, still more preferably 30, and particularly preferably 25. When the number of carbon atoms of the compound (A) falls within the above range, hydrophobicity of the directed self-assembling film can be more improved.

The compound (A) preferably has at least one selected from the group consisting of a structure represented by the following formula (1), a structure represented by the following formula (2) and a structure represented by the following formula (3).

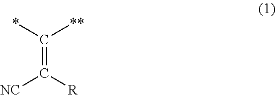

In the above formula (1), R represents —CN or —COOR¹, wherein R¹ represents a hydrogen atom or a monovalent hydrocarbon group having 1 to 6 carbon atoms; and * and ** each denote a site that bonds to a part other than the structure represented by the above formula (1) in the compound.

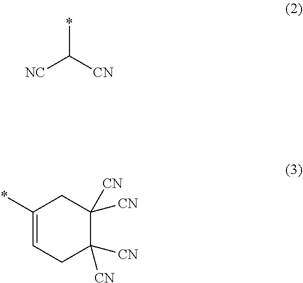 [SMILES: *C(C#N)C#N ; *C1=CCC(C#N)(C#N)C(C#N)(C#N)C1]

In the above formula (2), * denotes a site that bonds to a part other than the structure represented by the above formula (2) in the compound.

In the above formula (3), * denotes a site that bonds to a part other than the structure represented by the above formula (3) in the compound.

Examples of the monovalent hydrocarbon group having 1 to 6 carbon atoms which may be represented by R^{l} in the above formula (1) include:

chain hydrocarbon groups such as a methyl group, an ethyl group, a propyl group, and a butyl group;

alicyclic hydrocarbon groups such as a cyclopentyl group and a cyclohexyl group;

aromatic hydrocarbon groups such as a phenyl group; and the like.

In the compound (A), a part other than the structures represented by the above formulae (1) to (3) may have, for example,

a chain hydrocarbon group, e.g.,:

an alkyl group having 1 to 20 carbon atoms such as a methyl group, an ethyl group, a butyl group, a nonyl group or an undecyl group,

an alkenyl group having 2 to 20 carbon atoms such as an ethenyl group, a propenyl group, a butenyl group, a pentenyl group, or a hexenyl group, or

an alkynyl group having 2 to 20 carbon atoms such as an ethynyl group, a propynyl group, a butynyl group, a pentynyl group or a hexynyl group;

an aliphatic ring such as a cyclopentane ring or a cyclohexane ring;

an aromatic ring such as a benzene ring or a naphthalene ring; or the like.

Of these, the compound (A) has preferably the alkyl group having 1 to 20 carbon atoms, the alkenyl group having 2 to 20 carbon atoms, the aliphatic ring or the aromatic ring, and more preferably an alkyl group having 4 to 15 carbon atoms or an alkenyl group having 4 to 10 carbon atoms. When the compound (A) has the structure described above, hydrophobicity of the directed self-assembling film can be more improved.

Examples of the compound (A) include compounds represented by the following formulae (i-1) to (i-5) (hereinafter, may be also referred to as “compounds (i-1) to (i-5)”), and the like.

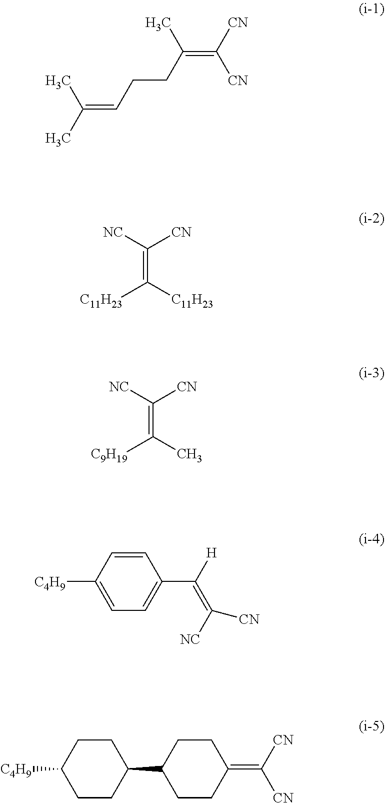

Moreover, the compound (A) is exemplified by compounds each having one cyano group, such as hexanenitrile, octanenitrile, decanenitrile, dodecanenitrile or tetradecanenitrile, and the like.

The compound (A) may be either a liquid or a solid at normal temperature (25° C.). In a case in which the compound (A) is a liquid at normal temperature, the lower limit of the vapor pressure at 150° C. of the compound (A) is preferably 0.1 Pa, more preferably 1 Pa, and still more preferably 5 Pa. The upper limit of the vapor pressure is, for example, 10⁵ Pa. When the vapor pressure of the compound (A) falls within the above range, evaporation of the compound (A) not interacting with the surface of the substrate is enabled in forming the directed self-assembling film, whereby the need for removing by washing or the like can be eliminated.

The lower limit of the content of the compound (A) with respect to the total components other than the solvent (B) in the directed self-assembling material is preferably 70% by mass, more preferably 80% by mass, and still more preferably 90% by mass. The content may be 100% by mass. One, or two or more types of the compound (A) may be used.

Synthesis Method of Compound (A)

In a case in which the compound (A) is a compound having the structure represented by the above formula (1), for example, the compound (A) may be synthesized by carrying out a dehydrative condensation reaction of ketone or aldehyde with malononitrile, cyano acetic acid or a cyano acetic acid ester in the presence of ammonium acetate, acetic acid and the like in a solvent such as toluene. Compounds (A) other than those represented by the above formula (1) can be synthesized by a well-known method.

(B) Solvent

The solvent (B) is not particularly limited as long as it is a solvent capable of dissolving or dispersing at least the compound (A) and the other optional component(s) which may be contained as needed.

The solvent (B) is exemplified by an alcohol solvent, an ether solvent, a ketone solvent, an amide solvent, an ester solvent, a hydrocarbon solvent, and the like.

Examples of the alcohol solvent include:

aliphatic monohydric alcohol solvents having 1 to 18 carbon atoms such as 4-methyl-2-pentanol and n-hexanol;

alicyclic monohydric alcohol solvents having 3 to 18 carbon atoms such as cyclohexanol;

polyhydric alcohol solvent having 2 to 18 carbon atoms such as 1,2-propylene glycol;

polyhydric alcohol partially etherated solvents having 3 to 19 carbon atoms such as propylene glycol monomethyl ether; and the like.

Examples of the ether solvent include:

dialkyl ether solvents such as diethyl ether, dipropyl ether, dibutyl ether, dipentyl ether, diisoamyl ether, dihexyl ether and diheptyl ether;

cyclic ether solvents such as tetrahydrofuran and tetrahydropyran;

aromatic ring-containing ether solvents such as diphenyl ether and anisole (methyl phenyl ether); and the like.

Examples of the ketone solvent include:

chain ketone solvents such as acetone, methyl ethyl ketone, methyl n-propyl ketone, methyl n-butyl ketone, diethyl ketone, methyl iso-butyl ketone (MIBK), methyl amyl ketone, ethyl n-butyl ketone, methyl n-hexyl ketone, di-iso-butyl ketone and trimethylnonanone;

cyclic ketone solvents such as cyclopentanone, cyclohexanone, cycloheptanone, cyclooctanone and methylcyclohexanone;

2,4-pentanedione, acetonylacetone, and acetophenone; and the like.

Examples of the amide solvent include:

cyclic amide solvents such as N,N′-dimethylimidazolidinone and N-methylpyrrolidone;

chain amide solvents such as N-methylformamide, N,N-dimethylformamide, N,N-diethylformamide, acetamide, N-methylacetamide, N,N-dimethylacetamide and N-methylpropionamide; and the like.

Examples of the ester solvent include:

acetic acid ester solvents such as ethyl acetate and n-butyl acetate;

lactic acid ester solvents such as ethyl lactate and n-butyl lactate;

polyhydric alcohol carboxylate solvents such as propylene glycol acetate;

polyhydric alcohol partially etherated carboxylate solvents such as propylene glycol monomethyl ether acetate;

lactone solvents such as γ-butyrolactone and δ-valerolactone;

polyhydric carboxylic acid diester solvents such as diethyl oxalate;

carbonate solvents such as dimethyl carbonate, diethyl carbonate, ethylene carbonate and propylene carbonate; and the like.

Examples of the hydrocarbon solvent include:

aliphatic hydrocarbon solvents having 5 to 12 carbon atoms such as n-pentane and n-hexane; aromatic hydrocarbon solvents having 6 to 16 carbon atoms such as toluene and xylene; and the like.

Of these, the solvent (B) is preferably the ester solvent and/or the ketone solvent, more preferably the polyhydric alcohol partially etherated carboxylate solvent and/or the chain ketone solvent, and still more preferably propylene glycol monomethyl ether acetate and/or methyl amyl ketone. The directed self-assembling material may contain one, or two or more types of the solvent (B).

Other Optional Component

The other optional component(s) is/are exemplified by a surfactant and the like. When the directed self-assembling material contains the surfactant, the coating characteristics on the base material surface may be improved.

Preparation Method of Directed Self-Assembling Material

The directed self-assembling material may be prepared by, for example, mixing the compound (A), the solvent (B), and as needed the other optional component(s) at a predetermined ratio, and preferably filtering the resulting mixture through a high-density polyethylene filter, etc., having fine pores of about 0.45 μm. The lower limit of the solid content concentration of the directed self-assembling material is preferably 0.1% by mass, more preferably 0.5% by mass, and still more preferably 1% by mass. The upper limit of the solid content concentration is preferably 30% by mass, more preferably 10% by mass, and still more preferably 5% by mass. The “solid content concentration” as referred to means a concentration (% by mass) of total components other than the solvent (B) in the directed self-assembling material.

### Forming Process of Directed Self-Assembling Film

A forming process of the directed self-assembling film includes a step of overlaying a film (hereinafter, may be also referred to as “overlaying step”) on the surface of a substrate having a first region which includes a metal atom (hereinafter, may be also referred to as “metal atom (a)”) in a surface layer thereof, using the directed self-assembling material. Since the directed self-assembling material is used in the forming process of the directed self-assembling film, convenient and highly selective hydrophobilization of the surface of the substrate having a region which includes a metal atom in the surface layer thereof is enabled, whereby the hydrophobilization treatment enables a superior blocking performance for metal oxide formation by an ALD process or a CVD process to be achieved. Hereinafter, the overlaying step will be described.

Overlaying Step

In this step, by using the directed self-assembling material, a film is overlaid on the surface of a substrate having a first region which includes the metal atom (a) in the surface layer thereof.

The substrate is exemplified by a metal substrate, and the like.

The metal atom (a) is not particularly limited as long as it is a metal element. It is to be noted that silicon is nonmetal and does not fall under the category of the metal atom (a). Examples of the metal atom (a) include copper, iron, zinc, cobalt, aluminum, tin, tungsten, zirconium, titanium, tantalum, germanium, molybdenum, ruthenium, gold, silver, platinum, palladium, nickel, and the like. Of these, copper, cobalt, tungsten or tantalum is preferred.

The metal atom (a) may be included in the surface layer of the metal substrate in for form of, for example, a metal simple substance, an alloy, an electrically conductive nitride, a metal oxide, a silicide or the like.

Examples of the metal simple substance include simple substances of metals such as copper, iron, cobalt, tungsten and tantalum, and the like.

Examples of the alloy include a nickel-copper alloy, a cobalt-nickel alloy, a gold-silver alloy, and the like.

Examples of the electrically conductive nitride include tantalum nitride, titanium nitride, iron nitride, aluminum nitride, and the like.

Examples of the metal oxide include tantalum oxide, aluminum oxide, iron oxide, copper oxide, and the liker.

Examples of the silicide include iron silicide, molybdenum silicide, and the like.

Of these, the metal simple substance or the electrically conductive nitride is preferred, and a copper simple substance, a cobalt simple substance, a tungsten simple substance or tantalum nitride is more preferred.

The surface layer of the substrate has: a first region (I) preferably including the metal atom (a); and a second region (II) not including the metal atom (a) but preferably substantially consisting of only a nonmetal atom (b).

The nonmetal atom (b) may be included in the second region (II) in the form of, for example, a nonmetal simple substance, a nonmetal oxide, a nonmetal nitride, a nonmetal oxynitride or the like.

Examples of the nonmetal simple substance include simple substances of silicon, carbon and the like.

Examples of the nonmetal oxide include silicon oxide, and the like.

Examples of the nonmetal nitride include SiNx, Si₃N₄, and the like.

Examples of the nonmetal oxynitride include SiON, and the like.

Of these, the nonmetal oxide is preferred, and silicon oxide is more preferred.

A mode of the arrangement of the first region (I) and the second region (II) on the surface layer of the substrate is not particularly limited, and is exemplified by surficial, spotted, striped, and the like in a planar view. The size of the first region (I) and the second region (II) is not particularly limited, and the regions may have an appropriate desired size.

The shape of the metal substrate is not particularly limited, and may be an appropriate desired shape such as platy and the like.

The overlaying procedure of the film is not particularly limited, and the film may be overlaid by: applying of the directed self-assembling material; PVD (Physical Vapor Deposition); CVD (Chemical Vapor Deposition); or the like. The application procedure of the directed self-assembling material is exemplified by spin-coating, and the like.

In a case in which the film is overlaid by the applying of the directed self-assembling material, the coating film provided by the applying may be heated or baked (hereinafter, may be also referred to as “heating, etc.”). Means for heating, etc. may be, for example, an oven, a hot plate, and the like. The lower limit of the temperature of the heating, etc., is preferably 80° C., more preferably 100° C., still more preferably 120° C., and particularly preferably 140° C. The upper limit of the temperature of the heating, etc., is preferably 400° C., more preferably 300° C., still more preferably 200° C., and particularly preferably 160° C. The lower limit of the time period of the heating, etc., is preferably 10 sec, more preferably 30 sec, still more preferably 60 sec, and particularly preferably 120 sec. The upper limit of the time period of the heating, etc., is preferably 120 min, more preferably 60 min, still more preferably 10 min, and particularly preferably 5 min.

In addition, the forming process of the directed self-assembling film may include after the overlaying step, a step of removing the film on a region other than the first region (hereinafter, may be also referred to as “removing step”). More specifically, for the purpose of removing the compound (A) not interacting with the surface of the substrate from the coating film after heating, the coating film before or after the heating may be rinsed with an organic solvent or the like. Examples of the organic solvent include those similar to the solvent exemplified as the solvent (B) in the directed self-assembling material. Of these, the polyhydric alcohol partially etherated carboxylate solvent such as propylene glycol monomethyl ether acetate is preferred. Accordingly, the directed self-assembling film is formed in which the compound (A) remains in the first region which includes the metal atom (a) in the surface layer of the substrate.

### Substrate Treatment Method

The substrate treatment method includes: a step of overlaying a film on a surface of a substrate having a first region which includes a metal atom in a surface layer thereof, using the directed self-assembling material of the aforementioned embodiment (overlaying step); after the overlaying step, a step of removing the film on a region other than the first region (removing step); after the removing step, a step of forming a pattern principally containing a metal oxide by an ALD process or a CVD process on the region other than the first region, of the surface of the substrate (hereinafter, may be also referred to as “forming step”). The substrate treatment method may include after the forming step, a step of removing the compound (A) that remains in the first region after the forming step (hereinafter, may be also referred to as “compound-removing step”). The term “pattern principally containing a metal oxide” as referred to herein means that the pattern may contain impurities in addition to the metal oxide. Each step will be described below.

Overlaying Step

In this step, a film is overlaid on the surface of a substrate having the first region which includes a metal atom in a surface layer thereof, by using the directed self-assembling material. This step is similar to the overlaying step in the forming process of the directed self-assembling film.

Removing Step

In this step, after the overlaying step, the film on a region other than the first region is removed. This step is similar to the removing step in the forming process of the directed self-assembling film.

Forming Step

In this step, after the removing step, a pattern principally containing a metal oxide is formed by an ALD (Atom Layer Deposition) process or a CVD (Chemical Vapor Deposition) process on a region other than the first region, on the surface of the substrate. The film on the first region (i.e., the directed self-assembling film) is formed with the directed self-assembling material, and therefore achieves superior blocking performance for metal oxide formation. Thus, selective formation of the pattern principally containing a metal oxide is enabled, on a region not provided with the directed self-assembling film on the surface of the substrate, i.e., a region other than the first region, more specifically, on a surface region other than the first region which includes the metal atom (a) in the surface layer of the substrate.

The CVD process is exemplified by various processes such as thermal CVD, plasma CVD, photo-induced CVD, vacuum CVD, laser CVD and organic metal CVD (MOCVD). The ALD process is exemplified by a thermal ALD process, a plasma ALD process, and the like.

Examples of the metal oxide that constitutes the pattern formed by the CVD process or the ALD process include oxides of one, or two or more types of metal selected from hafnium, aluminum, yttrium, zirconium, gallium, tungsten, titanium, tantalum, nickel, germanium, magnesium and the like. It is preferred that the pattern substantially consists of the metal oxide.

In CVD or ALD, examples of a precursor for use in forming a pattern including hafnium oxide include tetrakis(dimethylamido)hafnium, tetrakis(diethylamido)hafnium, bis(methyl-η⁵-cyclopentadienyl)dimethyl hafnium, bis(methyl-η⁵-cyclopentadienyl)methoxymethyl hafnium, and the like. Examples of other precursor include trimethyl aluminum, diethyl zinc, bis(methyl-η⁵-cyclopentadienyl)methoxymethyl zirconium, and the like.

The lower limit of the average thickness of the pattern formed is preferably 0.1 nm, more preferably 1 nm, and still more preferably 2 nm. The upper limit of the average thickness is preferably 500 nm, more preferably 100 nm, and still more preferably 50 nm.

Compound-Removing Step

In this step, the compound (A) that remains in the first region after the forming step is removed. The removing may be carried out by, for example, dry etching, wet etching or the like.

As the dry etching procedure, for example, a procedure performed using a well-known dry etching apparatus is exemplified. Examples of a source gas which may be used in the dry etching include: fluorinated gas such as CHF₃, CF₄, C₂F₆, C₃F₈ and SF₆; chlorinated gas such as Cl₂ and BCl₃; oxygen-based gas such as O₂ and O₃; reductive gas such as H₂, NH₃, CO, CO₂, CH₄, C₂H₂, C₂H₄, C₂H₆, C₃H₄, C₃H₆, C₃H₈, HF, HI, HBr, HCl, NO, NH₃ and BCl₃; inert gas such as He, N₂ and Ar; and the like. These gases may be used as a mixture. Of these, the oxygen-based gas is preferred.

In the wet etching, an etching liquid is used, and the etching liquid which may be used is exemplified by an acid, a base, a mixture of the same, and the like. Specific examples include an SC-1 washing liquid (mixture of aqueous ammonium hydroxide solution and hydrogen peroxide solution), an SC-2 washing liquid (mixture of aqueous hydrochloric acid solution and hydrogen peroxide solution), a piranha solution (mixture of sulfuric acid and hydrogen peroxide solution), and the like.

As described in the foregoing, a substrate can be obtained which is selectively patterned with principally a metal oxide in the surface region other than the first region which includes the metal atom (a) in the surface layer.

### Substrate Treatment System

The substrate treatment system includes: a mechanism for overlaying a film on a surface of a substrate having a first region which includes a metal atom in a surface layer thereof, using the directed self-assembling material of the above embodiment (hereinafter, may be also referred to as “overlaying mechanism”); a mechanism for removing the film on a region other than the first region (hereinafter, may be also referred to as “film-removing mechanism”) after the overlaying; and a mechanism for forming a pattern principally containing a metal oxide by an ALD process or a CVD process on the region other than the first region, of the surface of the substrate (hereinafter, may be also referred to as “forming mechanism”) after the removing.

Each mechanism will be described below.

Overlaying Mechanism

This mechanism is for overlaying a film on the surface of a substrate having a first region which includes a metal atom in a surface layer thereof, by using the directed self-assembling material of the above embodiment. The overlaying mechanism includes: a tank for storing the directed self-assembling material; a overlaying zone for overlaying a film on the surface of a substrate; and the like. The overlaying zone may include, for example: an applying zone for carrying out the step of applying the directed self-assembling material on the surface of the substrate; a heating zone for heating or baking the coating film provided by the applying step; and the like. This mechanism overlays the film from the directed self-assembling material in the region which includes the metal, of the surface of the substrate.

Film-Removing Mechanism

This mechanism is for removing the film on a region other than the first region, after the overlaying. The film-removing mechanism includes: a tank that stores a rinse agent such as an organic solvent for removing the film; a substrate-feeding zone that feeds the substrate having the film overlaid thereon; a rinse agent-supplying zone that supplies a rinse agent on the substrate fed; and the like. This mechanism serves to give the substrate having the directed self-assembling film being formed on which the compound (A) remains in the first region which includes the metal atom (a), on the surface of the substrate.

Forming Mechanism

This mechanism is for forming a pattern principally containing a metal oxide by an ALD process or a CVD process on a region other than the first region, on the surface of the substrate, after film-removing. The forming mechanism includes: a substrate-feeding zone for feeding the substrate having the directed self-assembling film being formed thereon; a forming zone for forming the pattern principally containing a metal oxide by an ALD process or a CVD process on the substrate fed; and the like. This mechanism forms a substrate having the pattern principally containing a metal oxide on a surface region other than the first region that includes the metal atom (a) and is provided with the directed self-assembling film, i.e., on a region other than the first region, on the surface of the substrate.

In addition, the substrate treatment system may further include a mechanism for well-known dry etching or wet etching, as a mechanism for removing the compound (A) that remains in the first region (compound-removing mechanism), after the forming step. It is to be noted that the overlaying mechanism, the film-removing mechanism, the forming mechanism and the compound-removing mechanism may be incorporated into each separate apparatus, or two or more of the mechanisms may be incorporated into a single apparatus.

### EXAMPLES

Hereinafter, the present invention is explained in detail by way of Examples, but the present invention is not in any way limited to these Examples. Measuring methods for physical properties are described below.

¹H-NMR and ¹³C-NMR Analyses

¹H-NMR and ¹³C-NMR analyses were carried out using a nuclear magnetic resonance apparatus (“JNM-EX400” available from JEOL, Ltd.).

### Synthesis of Compound (A)

### Synthesis Example 1

Into a 300-mL eggplant-shaped flask equipped with a Dean and Stark trap, 18.93 g of methylheptenone (150 mmol), 10.3 g of malononitrile (125 mmol), 0.96 g of ammonium acetate (12.5 mmol), 1.50 g of acetic acid (25 mmol) and 150 g of toluene were added, and the mixture was refluxed with heating at 110° C. for 2 hrs in a nitrogen atmosphere.

After completion of the reaction, the mixture was filtered through a folded filter paper, and the filtrate was washed three times with ultra pure water to remove the salt and the acid. The organic layer was recovered and water was removed therefrom with anhydrous magnesium sulfate, followed by concentration carried out with an evaporator. Thus obtained concentrate was distilled under reduced pressure to give 18.0 g of a compound represented by the following formula (A-1).

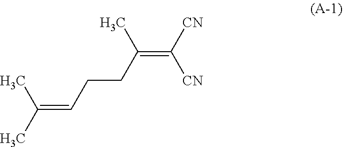

The boiling point, and the measurement data on ¹H-NMR and ¹³C-NMR of the compound (A-1) are shown below.

Boiling point: 103° C./15 Pa

¹H-NMR (δ/ppm) (CDCl₃): 5.00 (s, 1H, CH), 2.63 (br, 2H, CH₂), 2.24 (s, 5H, CH₂, CH₃), 1.67 (s, 3H, CH₃), 1.54 (s, 3H, CH₃)

¹³C-NMR (δ/ppm) (CDCl₃): 182 (═C<), 135 (>C═), 121 (CN), 111 ((CH₃)₂>C═), 86 (—CH═), 38 (CH₃), 25 (CH₂), 24 (CH₂), 22 (CH₃), 18 (CH₃)

### Synthesis Example 2

Into a 300-mL eggplant-shaped flask equipped with a Dean and Stark trap, 12.00 g of 12-tricosanone (35.4 mmol), 2.11 g of malononitrile (32 mmol), 0.25 g of ammonium acetate (3.2 mmol), 0.42 g of acetic acid (6.4 mmol) and 150 g of toluene were added, and the mixture was refluxed with heating at 110° C. for 2 hrs in a nitrogen atmosphere.

After completion of the reaction, the mixture was filtered through a folded filter paper, and the filtrate was washed three times with ultra pure water to remove the salt and the acid. The organic layer was recovered and water was removed therefrom with anhydrous magnesium sulfate, followed by filtration again through a folded filter paper and concentration of the filtrate carried out with an evaporator to give a solid.

Next, 100 g of cyclohexane was added to the solid thus obtained, which was dissolved by heating. Thereafter, the temperature was slowly lowered to normal temperature, whereby white crystals were precipitated. The crystals were recovered by vacuum filtration, and dried at normal temperature under a reduced pressure to give 9.83 g of a compound represented by the following formula (A-2).

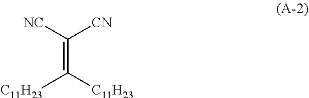

The melting point, and the measurement data on ¹H-NMR and ¹³C-NMR of the compound (A-2) are shown below.

Melting point: 68° C.

¹H-NMR (δ/ppm) (CDCl₃): 1.55 (br, 4H, CH₂), 1.25 (br, 36H, CH₂), 0.87 (t, 6H, CH₃)

¹³C-NMR (δ/ppm) (CDCl₃): 186 (═C<), 112 (CN), 85 (>C═), 42 (CH₂), 35 (CH₂), 32×2 (CH₂), 29×10 (CH₂), 28 (CH₂), 23 (CH₂), 22 (CH₂), 14 (CH₃)

### Synthesis Example 3

Into a 300-mL eggplant-shaped flask equipped with a Dean and Stark trap, 17.09 g of 2-undecanone (100 mmol), 6.28 g of malononitrile (95 mmol), 0.77 g of ammonium acetate (10 mmol), 1.20 g of acetic acid (20 mmol) and 150 g of toluene were added, and the mixture was refluxed with heating at 110° C. for 2 hrs in a nitrogen atmosphere.

After completion of the reaction, the mixture was filtered through a folded filter paper, and the filtrate was washed three times with ultra pure water to remove the salt and the acid. The organic layer was recovered and water was removed therefrom with anhydrous magnesium sulfate, followed by concentration carried out with an evaporator. Thus obtained concentrate was distilled under reduced pressure to give 15.3 g of a compound represented by the following formula (A-3).

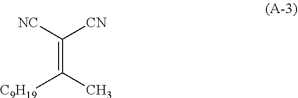

The boiling point and the measurement data on ¹H-NMR and ¹³C-NMR of the compound (A-3) are shown below.

Boiling point: 122° C./14 Pa

¹H-NMR (δ/ppm) (CDCl₃): 2.58 (t, 2H, CH₂), 2.27 (s, 3H, CH₃), 1.61 (m, 2H, CH₂), 1.32 (s, 12H, CH₂), 0.90 (t, 3H, CH₃)

¹³C-NMR (δ/ppm) (CDCl₃): 182 (═C<), 111 (CN), 85 (>C═), 38 (CH₂), 31 (CH₂), 29 (CH₂), 27 (CH₃), 22 (CH₂), 14 (CH₃)

### Synthesis Example 4

Into a 300-mL eggplant-shaped flask equipped with a Dean and Stark trap, 16.22 g of 4-butylbenzaldehyde (100 mmol), 5.61 g of malononitrile (85 mmol), 0.77 g of ammonium acetate (10 mmol), 1.20 g of acetic acid (20 mmol) and 150 g of toluene were added, and the mixture was refluxed with heating at 110° C. for 2 hrs in a nitrogen atmosphere.

After completion of the reaction, the mixture was filtered through a folded filter paper, and the filtrate was washed three times with ultra pure water to remove the salt and the acid. The organic layer was recovered and water was removed therefrom with anhydrous magnesium sulfate, followed by concentration carried out with an evaporator. Thus obtained concentrate was distilled under reduced pressure to give 14.3 g of a compound represented by the following formula (A-4).

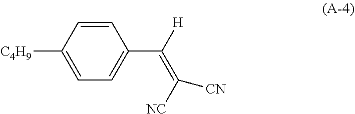

The boiling point, and the measurement data on ¹H-NMR and ¹³C-NMR of the compound (A-4) are shown below.

Boiling point: 140° C./7.3 Pa

¹H-NMR (6/ppm) (CDCl₃): 7.86 (d, 2H, Ph), 7.75 (s, 1H, Ph), 7.34 (d, 2H, Ph), 2.72 (m, 2H, CH₂), 1.65 (m, 2H, CH₂), 1.41 (m, 2H, CH₂), 1.33 (m, 3H, CH₃)

¹³C-NMR (δ/ppm) (CDCl₃): 159 (═C<), 151 (Ph—C═), 130, 129*3, 128 (Ph), 114, 113 (CN), 35.8 (CH₂), 33.2 (CH₂), 22.2 (CH₂), 13.8 (CH₃)

### Synthesis Example 5

Into a 300-mL eggplant-shaped flask equipped with a Dean and Stark trap, 20.00 g of 4-(trans-4-butylcyclohexyl)cyclohexanone (80 mmol), 4.40 g of malononitrile (67 mmol), 0.52 g of ammonium acetate (6.7 mmol), 0.80 g of acetic acid (13.4 mmol) and 150 g of toluene were added, and the mixture was refluxed with heating at 110° C. for 2 hrs in a nitrogen atmosphere.

After completion of the reaction, the mixture was filtered through a folded filter paper, and the filtrate was washed three times with ultra pure water to remove the salt and the acid. The organic layer was recovered and water was removed therefrom with anhydrous magnesium sulfate, followed by concentration carried out with an evaporator. The concentrate thus obtained was recrystallized in cyclohexane to give 14.5 g of a compound represented by the following formula (A-5).

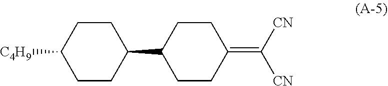

The melting point, and the measurement data on ¹H-NMR and ¹³C-NMR of the compound (A-5) are shown below.

Melting point: 86° C.

¹H-NMR (δ/ppm) (CDCl₃): 3.02 (d, 2H, CH), 2.31 (m, 2H, CH₂), 2.08 (m, 2H, CH₂), 1.78-1.55 (m, 4H, CH₂), 1.41-0.84 (m, 17H, CH₂, CH₃)

¹³C-NMR (δ/ppm) (CDCl₃): 185 (═C<), 112 (CN), 82.3 (>C═), 41.9*2 (CH), 37.6, 37.6, 37.2, 34.3, 33.2, 32.1, 31.0, 30.0, 26.6, 22.2 (CH₂), 14.1 (CH₃)

Compound (A-6): octadodecyl phosphate (Wako Pure Chemical Industries, Ltd., used directly)

### Preparation of Directed Self-Assembling Material

The solvent (B) used in preparing the directed self-assembling materials is as shown below.

(B) Solvent

B-1: propylene glycol monomethyl ether acetate (PGMEA)

B-2: methyl amyl ketone

### Example 1

A directed self-assembling material (S-1) was prepared by adding 40 g of (B-1) as the solvent (B) to 1.25 g of (A-1) as the compound (A), stirring the mixture and then filtering the mixture through a high-density polyethylene filter having a pore size of 0.45 μm.

### Examples 2 to 5 and Comparative Example 1

Directed self-assembling materials (S-2) to (S-6) were prepared in a similar manner to Example 1 except that each component of the type and the mass (g) shown in Table 1 below was used.

TABLE 1

| | | Mass (g) | | | | | |
|---|---|---|---|---|---|---|---|
| | | Example | Example | Example | Example | Example | Comparative |
| | | 1 | 2 | 3 | 4 | 5 | Example 1 |
| | | Directed self-assembling material | | | | | |
| | | S-1 | S-2 | S-3 | S-4 | S-5 | S-6 |
| (A) Compound | A-1 | 1.25 | | | | | |
| | A-2 | | 1.25 | | | | |
| | A-3 | | | 1.25 | | | |
| | A-4 | | | | 1.25 | | |
| | A-5 | | | | | 1.25 | |
| | A-6 | | | | | | 1.25 |
| (B) Solvent | B-1 | 40 | | 40 | 40 | | |
| | B-2 | | 40 | | | 40 | 40 |

| | | | | | | |
|---|---|---|---|---|---|---|
| Solid content concentration | 3 | 3 | 3 | 3 | 3 | 3 |
| (% by mass) | | | | | | |

### Formation of Directed Self-Assembling Film

An 8-inch copper substrate was immersed in a 5% by mass aqueous oxalic acid solution, and then dried under nitrogen flow to remove an oxide coating film on the surface thereof. A silicon oxide substrate was subjected to a surface treatment with isopropanol.

Next, each directed self-assembling material prepared as described above was spin-coated at 1,500 rpm by using a track (Tokyo Electron Limited, “TELDSA ACT8”), and baled at 150° C. for 180 sec. Thereafter, the coating film after the baking was rinsed under the following rinse conditions to form a directed self-assembling film.

Rinse conditions: Dynamic coating with PGMEA was conducted at the center of the substrate at 1,000 rpm for 5 sec, and thereafter spinning off was carried out at 750 rpm for 1 sec. After repeating the coating and spinning once again, spinning off was carried out at higher rotation frequency.

### Evaluations

Contact Angle

The contact angle of on the surface of the directed self-assembling film formed as described above was measured by using a contact angle meter (Kyowa Interface Science Co., LTD “Drop master DM-501”).

Evaluation of Metal Oxide Blocking

Evaluation of metal oxide blocking was carried out by using Cambridge Nanotech FIJI in Stanford University, under conditions shown in Table 2 below. As the precursor, tetrakis(dimethylamido)hafnium was used, and water was used as a catalytic promoter. ALD cycle was preset to be 47 cycles, and the presence/absence of formation of oxide layers on various coating films was determined.

TABLE 2

| Parameter | Value |
|---|---|
| Film type | ALD HfO₂ |
| Chamber | Cambridge Nanotech Fiji |
| Stage temp. | 200° C. |
| Hf precursor | Tetrakis(dimethylamido)hafnium |
| Co-reactant | H₂O |
| Recipe timing | 0.3 s Hf/15 s purge/0.06 s H₂O/23 s purge |
| #cycles | 47 or 22 cycles |
| Thickness on Si | 5.4 nm or 2.8 nm (by ellipsometry) |
| Loading | Let stage cool for 15 mins before loading |
| Queue Time | <3 hours from pick-up |

Quantitative determination was carried out for Hf components on the coating film after the ALD cycle, by an ESCA analysis. In ESCA, quantitative determination of the Hf components in terms of Hf₄f was carried out through excluding coating film components and substrate components from a 100 μφ up condition with “Quantum 200” (ULVAC, Inc.), and then the percentage was calculated, which was defined as “HfO₂ blocking rate”. A smaller value of the “HfO₂ blocking rate” means that the film has a superior Hf blocking performance.

TABLE 3

| | Directed self- | | | |
|---|---|---|---|---|
| | assembling | | | HfO₂ blocking rate |
| | material | SiO₂ | Cu | (wt %) |
| Blank | — | 36 | <10 | |
| Example 1 | S-1 | 38 | 67 | 50 |
| Example 2 | S-2 | 42 | 102 | 80 |
| Example 3 | S-3 | 39 | 86 | 75 |
| Example 4 | S-4 | 40 | 87 | 63 |
| Example 5 | S-5 | 39 | 93 | 77 |
| Comparative | S-6 | 52 | 96 | 44 |
| Example 1 | | | | |

As is seen from the results shown in Table 3 above, the directed self-assembling materials and the substrate treatment methods of Examples enable the surface of the substrate having a region which includes the metal atom in the surface layer thereof to be conveniently and highly selectively hydrophobilized, The hydrophobilization treatment enables a superior blocking performance for metal oxide formation by an ALD process to be achieved.

The directed self-assembling material of the embodiment of the present invention is capable of conveniently and highly selectively hydrophobilizing the surface of the substrate having a region which includes a metal atom in the surface layer thereof, whereby the hydrophobilization treatment enables a superior blocking performance for metal oxide formation by an ALD process or a CVD process to be achieved. The substrate treatment method and the substrate treatment system of the embodiments of the present invention enable a treatment for selectively modifying a substrate surface to executed out through achieving superior blocking performance for metal oxide formation in a hydrophobilized region by an ALD process or a CVD process. Therefore, the substrate treatment method, substrate treatment system and directed self-assembling material can be suitably used for working processes of semiconductor devices, and the like, in which microfabrication is expected be further in progress hereafter.

Obviously, numerous modifications and variations of the present invention are possible in light of the above teachings. It is therefore to be understood that within the scope of the appended claims, the invention may be practiced otherwise than as specifically described herein.

What is claimed is:

1. A substrate treatment method comprising:
overlaying a film on a surface of a substrate which comprises a first region comprising a metal atom in a surface layer thereof, using a directed self-assembling material which comprises a compound having no less than 6 carbon atoms and comprising at least one cyano group;
after the overlaying, removing the film on a region other than the first region; and
after the removing, forming a pattern principally comprising a metal oxide by an Atomic Layer Deposition process or a Chemical Vapor Deposition process on the region other than the first region, of the surface of the substrate.

2. The substrate treatment method according to claim 1, wherein the compound comprises a structure represented by formula (1), a structure represented by formula (2), a structure represented by formula (3), or a combination thereof, 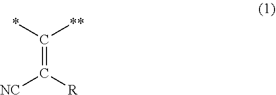
wherein,
in the formula (1), R represents —CN or —COOR¹, wherein R¹ represents a hydrogen atom or a monovalent hydrocarbon group having 1 to 6 carbon atoms; and * and ** each denote a site that bonds to a part other than the structure represented by the formula (1) in the compound, 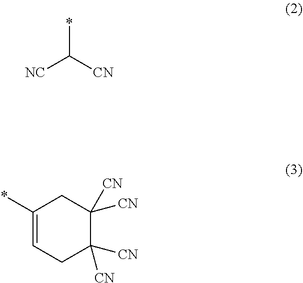 [SMILES: *C(C#N)C#N ; *C1=CCC(C#N)(C#N)C(C#N)(C#N)C1]
in the formula (2), * denotes a site that bonds to a part other than the structure represented by the formula (2) in the compound, and
in the formula (3), * denotes a site that bonds to a part other than the structure represented by the formula (3) in the compound.

3. The substrate treatment method according to claim 1, further comprising after the forming of the pattern, removing the compound that remains in the first region.

4. The substrate treatment method according to claim 1, wherein the overlaying comprises applying the directed self-assembling material on the surface of the substrate.

5. A substrate treatment system comprising:
a mechanism for overlaying a film on a surface of a substrate which comprises a first region comprising a metal atom in a surface layer thereof, using a directed self-assembling material which comprises a compound having no less than 6 carbon atoms and comprising at least one cyano group;
a mechanism for removing the film on a region other than the first region, after the overlaying; and
a mechanism for forming a pattern principally comprising a metal oxide by an Atomic Layer Deposition process or a Chemical Vapor Deposition process on the region other than the first region, of the surface of the substrate, after the removing.

6. A directed self-assembling material which comprises a compound having no less than 6 carbon atoms and comprising at least one cyano group.

7. A directed self-assembling material according to claim 6 which further comprises a solvent.

8. The directed self-assembling material according to claim 6, wherein the compound comprises a structure represented by formula (1), a structure represented by formula (2), a structure represented by formula (3), or a combination thereof, 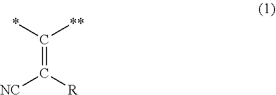
wherein,
in the formula (1), R represents —CN or —COOR¹, wherein R¹ represents a hydrogen atom or a monovalent hydrocarbon group having 1 to 6 carbon atoms; and * and ** each denote a site that bonds to a part other than the structure represented by the formula (1) in the compound, 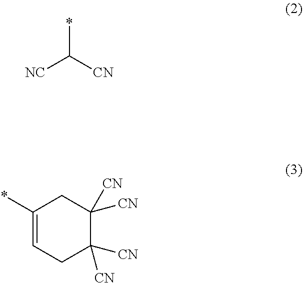 [SMILES: *C(C#N)C#N ; *C1=CCC(C#N)(C#N)C(C#N)(C#N)C1]
in the formula (2), * denotes a site that bonds to a part other than the structure represented by the formula (2) in the compound, and
in the formula (3), * denotes a site that bonds to a part other than the structure represented by the formula (3) in the compound.
